(19) United States
(12) Patent Application Publication
(10) Pub. No.: US 2024/0398849 A1
(43) Pub. Date: Dec. 5, 2024

(54) METHODS FOR THE TREATMENT OF HYPERTRIGLYCERIDEMIA WITH CYCLODEXTRINS

(71) Applicant: BEREN THERAPEUTICS P.B.C., West Hollywood, CA (US)

(72) Inventor: Jason CAMM, West Hollywood, CA (US)

(21) Appl. No.: 18/801,161

(22) Filed: Aug. 12, 2024

Related U.S. Application Data
(63) Continuation of application No. PCT/IB2023/051467, filed on Feb. 17, 2023.
(60) Provisional application No. 63/311,536, filed on Feb. 18, 2022.

(51) Int. Cl. A61K 31/724; A61P 3/10

(52) U.S. Cl. CPC A61K 31/724; A61P 3/10

(57) ABSTRACT

Disclosed herein are methods treating hypertriglyceridemia by administering a therapeutically effective amount of 2-hydroxypropyl-beta-cyclodextrin to the subject. In some cases, the therapeutically effective amount is an amount effective to decrease the amount of serum triglyceride by at least 10% after the administering as compared to prior to the administering.

### CROSS-REFERENCE

This application is a continuation of PCT application no. PCT/IB2023/051467, filed Feb. 17, 2023, which claims priority to U.S. provisional application No. 63/311,536, filed Feb. 18, 2022, which applications are herein incorporated by reference in their entirety.

### BACKGROUND

Hypertriglyceridemia is a disease that results in the presence of high amounts of triglycerides in the blood. Hypertriglyceridemia occurs in various physiologic conditions and in various diseases, and high triglyceride levels are associated with atherosclerosis, heart disease, and pancreatitis. Hypertriglyceridemia is a heavy burden to our modern society. Preclinical data suggest 2-hydroxypropyl-beta-cyclodextrins could have profound beneficial effects on the pathomechanisms responsible for hypertriglyceridemia disease development and arrest or reverse the progression of hypertriglyceridemia. Therefore, 2-hydroxypropyl-beta-cyclodextrins may provide a novel treatment option for hypertriglyceridemia.

### SUMMARY OF THE DISCLOSURE

There is a need for safe and effective treatments for hypertriglyceridemia. This disclosure addresses this unmet need.

The disclosure provides a method of treating hypertriglyceridemia in a subject, the method comprising: administering a therapeutically effective amount of 2-hydroxypropyl-beta-cyclodextrin to the subject to treat hypertriglyceridemia and/or reduce or prevent symptoms of hypertriglyceridemia. In some aspects, the hypertriglyceridemia is caused by high triglyceride levels in the subject, overeating, obesity, diabetes and/or insulin resistance, excessive alcohol consumption, kidney failure, nephrotic syndrome, genetic predisposition, lipoprotein lipase deficiency, lysosomal acid lipase deficiency, hypothyroidism, lupus, glycogen storage disease, propofol, and/or HIV medication. In one aspect, this disclosure provides a method of reducing symptoms of or inhibiting the development of hypertriglyceridemia in a subject, the method comprising administering a therapeutically effective amount of 2-hydroxypropyl-beta-cyclodextrin to the subject, the therapeutically effective amount being: (a) an amount effective to increase a circulating and/or systemic level of one or more oxysterol in the subject by at least about 10% after the administering as compared to prior to the administering; (b) an amount effective to increase plasma cholesterol crystal dissolution capacity (CCDC) by at least about 10% after the administering as compared to prior to the administering; (c) an amount effective to increase a level of ABCA1 and/or ABCG1 by at least about 10% after the administering as compared to prior to the administering; (d) about 50 mg/kg to about 2,000 mg/kg; (c) an amount effective to decrease the amount of serum triglyceride by at least 10% after the administering as compared to prior to the administering; or (f) any combination thereof, thereby reducing symptoms of or inhibiting the development of hypertriglyceridemia subject. In some aspects, the therapeutically effective amount is about 2 g to about 250 g of the 2-hydroxypropyl-beta-cyclodextrin.

In some aspects, the therapeutically effective amount is an amount sufficient to achieve a serum, plasma, and/or whole blood concentration of 2-hydroxpropyl-beta-cyclodextrin of about 0.6 mM to about 3 mM. In some aspects, the therapeutically effective amount is an amount effective to increase a circulating and/or systemic level of one or more oxysterol in the subject by at least about 10% after the administering as compared to prior to the administering. In some aspects, the circulating and/or systemic levels comprise serum, plasma, and/or whole blood levels. In some aspects, the one or more oxysterols are selected from the group consisting of: 27-hydroxycholesterol and 24-hydroxycholesterol. In some aspects, the at least about 10% comprises at least about 15%, at least about 20%, at least about 30%, at least about 40%, or at least about 50%.

In some aspects, the therapeutically effective amount is an amount effective to increase a circulating and/or systemic level of one or more oxysterol to about 40 ng/ml or greater. In some aspects, the therapeutically effective amount is an amount effective to increase a circulating and/or systemic level of one or more oxysterol to at least about 40 ng per mg of total circulating and/or systemic cholesterol. In some aspects, the one or more oxysterol comprises 27-hydroxycholesterol. In some aspects, the therapeutically effective amount is an amount effective to increase the circulating and/or systemic level of 27-hydroxycholesterol to at least about 100 ng/mL. In some aspects, the therapeutically effective amount is an amount effective to increase the circulating and/or systemic level of 27-hydroxycholesterol to at least about 90 ng per mg of total circulating and/or systemic cholesterol. In some aspects, the therapeutically effective amount is an amount sufficient to sustain the circulating and/or systemic level of the one or more oxysterol for at least 24 hours. In some aspects, the therapeutically effective amount is an amount effective to increase plasma cholesterol crystal dissolution capacity (CCDC) by at least about 10% after the administering as compared to prior to the administering. In some aspects, the therapeutically effective amount is an amount effective to increase a level of ABCA1 and/or ABCG1 by at least about 10% at after the administering as compared to prior to the administering. In some aspects, the therapeutically effective amount is an amount effective to decrease the amount of serum triglyceride by at least 10% after the administering as compared to prior to the administering. In some aspects, the therapeutically effective amount is about 50 mg/kg to about 2,000 mg/kg. In some aspects, the therapeutically effective amount is at least about 100 mg/kg. In some aspects, the therapeutically effective amount is at least about 250 mg/kg. In some aspects, the therapeutically effective amount is at least about 500 mg/kg. In some aspects, the therapeutically effective amount is at least about 1,000 mg/kg. In some aspects, the therapeutically effective amount is at least about 1,500 mg/kg. In some aspects, the therapeutically effective amount is about 500 mg/kg to about 1,500 mg/kg or about 800 mg/kg to about 1,200 mg/kg. In some aspects, the subject is an human individual.

In another aspect of the present invention, the administering further comprises: (i) administering, at a first time point, a therapeutically effective first dose of 2-hydroxypropyl-beta-cyclodextrin to the subject; and (ii) administering, at a second time point, a therapeutically effective second dose of 2-hydroxypropyl-beta-cyclodextrin to the subject. In some aspects, the second time point is at least 1 week after the first time point. In some aspects, the second time point is at least 2 weeks after the first time point. In some aspects, the second time point is at least one month after the first time point. In some aspects, the treating comprises decreasing or preventing progression and/or development of hypertriglyceridemia in the subject. In some aspects, the treating comprises mediating the regression of elevated serum triglyceride level in the subject. In some aspects, the treating results in one or more of the following: a) liver enzyme (e.g., ALT, AST) levels less than 2.5 times to normal; b) serum creatinine levels less than 0.3 mg/dl; or c) no substantial loss of sensorineural hearing. In some aspects, the administering is by intravenous administration.

Provided herein in various aspects, is a pharmaceutical composition comprising: an amount of 2-hydroxypropyl-beta-cyclodextrin effective to treat hypertriglyceridemia in a subject, and a pharmaceutically acceptable excipient. In some aspects, a pharmaceutical composition comprising: about 4 g to about 250 g of 2-hydroxypropyl-beta-cyclodextrin and a pharmaceutically acceptable excipient. In some aspects, the amount of 2-hydroxypropyl-beta-cyclodextrin is an amount effective to increase a circulating and/or systemic level of one or more oxysterol in the subject by at least about 10% after administering the pharmaceutical composition to the subject. In some aspects, the amount of 2-hydroxypropyl-beta-cyclodextrin is an amount effective to increase plasma cholesterol crystal dissolution capacity (CCDC) by at least about 10% after administering the pharmaceutical composition to the subject. In some aspects, the amount of 2-hydroxypropyl-beta-cyclodextrin is an amount effective to increase a level of ABCA1 and/or ABCG1 by at least about 10% after administering the pharmaceutical composition to the subject. In some aspects, the pharmaceutical composition is formulated for single dose administration. In some aspects, the pharmaceutical composition is formulated for intravenous administration.

In another aspect, this disclosure provides a kit comprising: (a) one or more container; and (b) the pharmaceutical composition disclosed herein, and the pharmaceutical composition is contained within the one or more container. In some aspects, the kit further comprises (c) instructions for use of the pharmaceutical composition for the treatment of hypertriglyceridemia in a subject and/or reducing or inhibiting the development of hypertriglyceridemia or symptoms thereof in a subject. In some aspects, at least one of the one or more container is an IV infusion bag. In some aspects, the one or more container comprises a single container comprising the pharmaceutical composition and one or more additional active pharmaceutical ingredients. In some aspects, the one or more container comprises a first container containing the pharmaceutical composition and a second container containing one or more additional active pharmaceutical ingredients. In some aspects, the kit further comprises one or more additional components selected from the group consisting of: an IV infusion bag, a catheter, tubing, a needle, a syringe, a solution, and any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings of which:

FIG. 1A depicts a non-limiting example of administration details of 2-hydroxypropyl-beta-cyclodextrin (HPBCD) to Western diet (WD)-fed mouse model.

FIG. 1B depicts a non-limiting example of administration details of HPBCD to Normal chow diet (NC)-fed mouse model.

FIG. 2A depicts an exemplary graph of triglyceride levels obtained from Western diet (WD)-fed mouse model treated with 2 g/kg HPBCD as a function of period after the administration of HPBCD.

FIG. 2B depicts an exemplary of triglyceride levels obtained from Normal chow diet (NC)-fed mouse model treated with 2 g/kg HPBCD as a function of period after the administration of HPBCD.

FIG. 3A depicts a graph of triglyceride levels obtained from volunteers administered 50 mg/kg, 250 mg/kg, 500 mg/kg, 1000 mg/kg or 1500 mg/kg HPBCD immediately prior to injection and 24 hours and 48 hours after the administration of HPBCD.

FIG. 3B depicts a graph of average change in triglyceride levels obtained from volunteers following administration of placebo, 500 mg/kg, 1000 mg/kg or 1500 mg/kg HPBCD at 24 hours and 48 hours after administration.

FIG. 3C depicts a graph of average change in triglyceride levels obtained from volunteers with baseline triglyceride levels greater than 100 mg/dL following administration of placebo, 500 mg/kg, 1000 mg/kg or 1500 mg/kg HPBCD at 24 hours and 48 hours after administration.

FIG. 3D depicts a graph of average change in triglyceride levels obtained from volunteers with baseline triglyceride levels less than or equal to 100 mg/dL following administration of placebo, 500 mg/kg, 1000 mg/kg or 1500 mg/kg HPBCD at 24 hours and 48 hours after administration.

### DETAILED DESCRIPTION OF THE DISCLOSURE

Disclosed herein are methods for the treatment of hypertriglyceridemia. In some aspects, the methods may involve administering a therapeutically effective amount of a cyclodextrin to a subject in need thereof (e.g., a subject having, suspected of having, or at risk of developing hypertriglyceridemia. In some cases, the therapeutically effective amount is an amount effective to increase a circulating and/or systemic level of one or more sterol and/or oxysterol in the subject compared to a baseline (e.g., pre-treatment with cyclodextrins). In a particular aspect, the cyclodextrin is 2-hydroxypropyl-beta-cyclodextrin.

In some aspects, disclosed herein are methods for reducing the amount of and/or the size of, and/or changing the shape of circulating (e.g., blood, plasma, serum) cholesterol crystals (and/or clots comprising cholesterol crystals) in an individual (e.g., a human) suffering from or expected to have or develop hypertriglyceridemia.

In some aspects, disclosed herein are methods for preventing or reducing the risk of developing hypertriglyceridemia in an individual (e.g., a human). Further disclosed herein are methods for preventing or reducing the risk of an increase in the amount of serum triglycerides in an individual (e.g., a human suffering from or suspected to suffer from hypertriglyceridemia). In a particular aspect, the cyclodextrin is 2-hydroxypropyl-beta-cyclodextrin.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “about” a number refers to that number plus or minus 10% of that number. The term “about” a range refers to that range minus 10% of its lowest value and plus 10% of its greatest value.

As used herein, the terms “subject,” “individual”, and “patient” are used interchangeably. None of the terms are to be interpreted as requiring the supervision of a medical professional (e.g., a doctor, nurse, physician's assistant, orderly, hospice worker). As used herein, the subject may be any animal, including mammals (e.g., a human or non-human animal) and non-mammals. In one embodiment, the subject is a human.

As used herein, the terms “treat,” “treating”, or “treatment,” and other grammatical equivalents, include ameliorating or preventing the underlying causes of one or more symptoms of a disease or condition; alleviating, abating, or ameliorating one or more symptoms of a disease or condition; ameliorating, preventing, or reducing the appearance, severity, or frequency of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. Methods of treatment as disclosed herein include disclosures of the use of the (e.g., pharmaceutical) compositions provided herein for the treatment of any indication described herein, and include disclosures of the (e.g., pharmaceutical) compositions provided herein for the use in treating any indication described herein.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier, or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

“Pharmaceutically acceptable excipient” as used herein, refers to any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The terms “effective amount” or “therapeutically effective amount,” as used herein, refer to a sufficient amount of an agent or a compound being administered which relieves, to some extent, one or more of the symptoms of the disease or condition being treated, or reduces the underlying cause of the disease or condition being treated. In some aspects, the result is a reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms or underlying cause of the disease (e.g., without undue adverse side effects). In some aspects, an appropriate “effective amount” in any individual case is determined using techniques, such as a dose escalation study. The term “therapeutically effective amount” includes, for example, a prophylactically effective amount. An “effective amount” of a compound disclosed herein may be an amount effective to achieve a desired effect or therapeutic improvement (e.g., without undue adverse side effects). An “effective amount” of a compound disclosed herein may be an amount effective to achieve one or more desired outcomes (e.g., a systemic and/or circulating level of a sterol or an oxysterol as described herein). It should be understood that, in some cases, “an effective amount” or “a therapeutically effective amount” varies from subject to subject, due to variation in metabolism of the composition, age, weight, general condition of the subject, concomitant medications the subject may be taking, the condition being treated, the severity of the condition being treated, and the judgment of the prescribing physician. In some instances, the disease or condition being treated is hypertriglyceridemia. The underlying cause of hypertriglyceridemia may be, e.g., high triglyceride levels in the subject, overeating, obesity, diabetes and/or insulin resistance, excessive alcohol consumption, kidney failure, nephrotic syndrome, genetic predisposition, lipoprotein lipase deficiency, lysosomal acid lipase deficiency, hypothyroidism, lupus, glycogen storage disease, propofol, HIV medication, and/or ischemia.

### Methods of Treating Hypertriglyceridemia

Disclosed herein are methods for treating a subject having, suspected of having, or at risk of developing hypertriglyceridemia. In some cases, treating a subject as described herein may inhibit, prevent, or reduce the development of elevation of serum triglyceride levels in the subject. In some cases, treating a subject as described herein may mediate, promote, enhance, or increase the regression of already-developed hypertriglyceridemia. In some cases, treating a subject as described herein may result in lower serum triglyceride levels in the subject. In some cases, reducing serum triglyceride levels in the subject may ameliorate, prevent, or reduce one or more symptoms associated with hypertriglyceridemia.

In various aspects, the methods involve administering a cyclodextrin to a subject. Cyclodextrins are a family of cyclic oligosaccharides, consisting of a cyclic (e.g., macrocyclic) ring of glucose subunits joined by α-1,4 glycosidic bonds. Cyclodextrins contain a number of glucose monomers in a ring formation. Common cyclodextrins include alpha-cyclodextrins (consisting of six glucose monomers), beta-cyclodextrins (consisting of seven glucose monomers), gamma-cyclodextrins (consisting of eight glucose monomers), and delta-cyclodextrins (consisting of nine glucose monomers). The outer portion of the ring structure is hydrophilic and the inner cavity of the ring structure is hydrophobic; thus, cyclodextrins generally are water soluble (e.g., due to the hydrophilic exterior), and capable of incorporating hydrophobic molecules in the cavity (e.g., due to the hydrophobic cavity). Parent cyclodextrins have limited water solubility; therefore, several chemically modified cyclodextrins have been synthesized where the hydroxyl groups are substituted with other chemical moieties to increase solubility. In various aspects, the methods provided herein involve administering a cyclodextrin to a subject (e.g., a human) in need thereof (e.g., having, suspected of having, or at risk of developing hypertriglyceridemia). In some cases, the subject has, is suspected of having, or is at risk of developing atherosclerotic plaques (e.g., cholesterol-rich plaques and/or lipid-rich plaques).

In particular aspects, the cyclodextrin is 2-hydroxypropyl-beta-cyclodextrin. In some instances, the 2-hydroxypropyl-beta-cyclodextrin is selected from the group consisting of: Kleptose® HP Parenteral Grade (Roquette Frères, #346114; accessible at roquette.com/-/media/roquette-sharepoint-libraries/sdol_product-specification-sheet/roquette_quality_specification-sheet_kleptose-hp-parenteral-grade_50_346114_en.pdf as of Aug. 26, 2020), Kleptose® HPB Parenteral Grade (Roquette Frères, #346111; accessible at roquette.com/-/media/roquette-sharepoint-libraries/sdol_product-specification-sheet/roquette_quality_specification-sheet_kleptose-hpb-parenteral-grade_50_346111_en.pdf as of Aug. 26, 2020), Kleptose® HPB-LB Parenteral Grade (Roquette Frères, #346115; accessible at roquette.com/-/media/roquette-sharepoint-libraries/sdol_product-specification-sheet/roquette_quality_specification-sheet_kleptose-hpb-lb-parenteral-grade_50_346115_en.pdf as of Aug. 26, 2020), Cavitron® W7 HP5 Pharma cyclodextrin (Ashland; accessible at ashland.com/file_source/Ashland/Product/Documents/Pharmaceutical/PC_11734_Cavitron_Cav asol.pdf as of Aug. 26, 2020), Cavitron® W7 HP7 Pharma cyclodextrin (Ashland; accessible at ashland.com/file_source/Ashland/Product/Documents/Pharmaceutical/PC_11734_Cavitron_Cav asol.pdf as of Aug. 26, 2020), Trappsol® Cyclo™ (Cyclo Therapeutics, Inc.; accessible at cyclotherapeutics.com/cyclodextrins/trappsol-cyclo as of Aug. 26, 2020), and VTS-270/adrabetadex.

In certain aspects, a cyclodextrin provided or used in a (e.g., pharmaceutical) composition or method or other application herein is a mixture of cyclodextrins; for example, in some aspects, a 2-hydroxypropyl-beta-cyclodextrin provided herein comprises a mixture of 2-hydroxypropyl-beta-cyclodextrins. In some aspects, a cyclodextrin molecule provided herein is optionally substituted with one or more chemical group, each chemical group independently being a hydroxypropyl group, a hydroxyethyl group, a methyl group, an ethyl group, a carboxymethyl group, a heptakis (2,6-di-O-methyl) group, a sulfoethyl group, a sulfopropyl group, and/or a sulfobutyl ethyl group, or its oligomer thereof. In some preferred aspects, the cyclodextrin is a hydroxypropyl-beta-cyclodextrin, such as wherein one or more hydroxyl of the cyclodextrin is substituted with hydroxypropyl (e.g., 2-hydroxypropyl group). For example, one or more hydroxyl positions are substituted by one or more hydroxypropyl groups by substituting the H of the hydroxyl (OH) with a —CH₂CH₂ (OH)CH₃ group, such as illustrated in Formula I below. In some aspects, the 2-hydroxypropyl-beta-cyclodextrin comprises a plurality of cyclodextrins with various different Degree of Substitution (DS) values and/or having a Molar Substitution (MS) value.

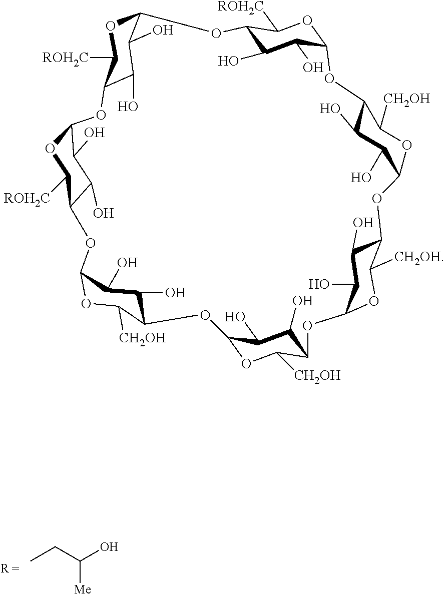

wherein each R is independently H or as noted above, and wherein at least one R is not H.

### Formula I

In some aspects, the plurality of beta-cyclodextrin molecules in a beta-cyclodextrin (mixture of beta-cyclodextrin molecules) is characterized by an average molar substitution. The “molar substitution,” or “MS,” is the average number of substituents per glucose unit in the beta-cyclodextrin molecules. In some aspects, MS is determined according to the procedures set forth in the USP monograph on Hydroxypropyl Betadex (USP NF 2015) (“USP Hydroxypropyl Betadex monograph”), incorporated herein by reference in its entirety. In some aspects, the (e.g., pharmaceutical) compositions provided herein contain a plurality of beta-cyclodextrin molecules having an average MS of at least about 0.3. In some aspects, the (e.g., pharmaceutical) compositions provided herein contain a plurality of beta-cyclodextrin molecules having an average MS of about 0.3 to 1.2. In some aspects, the (e.g., pharmaceutical) compositions provided herein contain a plurality of beta-cyclodextrin molecules having an average MS of 0.8-1.2.

In some aspects, the plurality of beta-cyclodextrin molecules is characterized by average degree of substitution. The term “degree of substitution,” or “DS,” refers to the total number of substituents substituted directly or indirectly on a beta-cyclodextrin molecule. In some aspects, the beta-cyclodextrin molecule may have one, or multiple, glucose units that are substituted by a substituent at a hydroxyl position. Thus, average DS refers to the total number of substituents in a population of beta-cyclodextrins divided by the number of beta-cyclodextrin molecules. In some aspects, the average DS of the molecule is measured using Electron Spray Ionization-Mass Spectrometry (ESI-MS) analysis (e.g., HPLC-ESI-MS, etc.). In some aspects, the average DS of the molecule is determined by peak heights of an electrospray MS spectrum. In some aspects, the average DS of the molecule is determined by multiplying the MS by 7. In some aspects, the (e.g., pharmaceutical) compositions provided herein contain a plurality of beta-cyclodextrin molecules having an average DS of about 2.0 to 9.0. In some aspects, the (e.g., pharmaceutical) compositions provided herein contain a plurality of beta-cyclodextrin molecules having an average DS of about 6.0 to 8.0.

In some aspects, any atom of the cyclodextrins described herein (e.g., 2-hydroxypropyl-beta-cyclodextrin) may be substituted with any suitable isotope. In a particular embodiment, any one or more hydrogen atoms of the cyclodextrins described herein (e.g., 2-hydroxypropyl-beta-cyclodextrin) may be substituted or replaced with deuterium atoms. Such cyclodextrins are expected to have similar or improved properties as compared to the original cyclodextrin that does not contain deuterium. Deuterium is a safe, stable, non-radioactive isotope of hydrogen. Compared to hydrogen, deuterium forms stronger bonds with carbon. In some instances, the increased bond strength imparted by deuterium can positively impact properties of the cyclodextrins, creating the potential for improved drug efficacy, safety, and/or tolerability. In addition, deuteration may cause decreased metabolic clearance in vivo, thereby increasing the half-life and circulation of the compound. At the same time, because the size and shape of deuterium are essentially identical to those of hydrogen, replacement of hydrogen by deuterium would not be expected to affect the biochemical potency and selectivity of the compound as compared to the original chemical entity that contains only hydrogen.

In various aspects, a therapeutically effective amount of 2-hydroxypropyl-beta-cyclodextrin is administered to the subject. In some aspects, administration of a therapeutically effective amount of 2-hydroxypropyl-beta-cyclodextrin increases a circulating and/or systemic level of one or more derivative of cholesterol as compared to a baseline. In some aspects, the one or more derivative of cholesterol is a by-product of cholesterol biosynthesis. In some aspects, the one or more derivative of cholesterol comprises a hydrogenated product, products with differently hydrogenated 1H-cyclopenta[a]phenanthren-3-ol products, or products formed with a hydroxyl, epoxyl, or keto group. In some cases, the one or more derivative of cholesterol is an oxysterol or a sterol.

A therapeutically effective amount may be an amount of 2-hydroxypropyl-beta-cyclodextrin effective to increase a circulating and/or a systemic amount of one or more sterols and/or oxysterols in the subject as compared to a baseline. A circulating and/or systemic amount of a sterol and/or oxysterol may be an amount present in a biological sample of the subject (e.g., blood (e.g., whole blood), plasma, serum, and the like). In some cases, the level of a circulating and/or systemic sterol and/or oxysterol may be increased by at least about 10% as compared to a baseline (e.g., at 24 hours after treatment), (e.g., at least about 10%, at least about 15%, at least about 20%, at least about 25%, at least about 30%, at least about 35%, at least about 40%, at least about 45%, at least about 50%, or greater). In some cases, the level of a circulating and/or systemic sterol and/or oxysterol may be increased to at least about 40 ng/ml (e.g., at least about 40 ng/mL, at least about 50 ng/ml, at least about 60 ng/mL, at least about 70 ng/mL, at least about 80 ng/mL, at least about 90 ng/ml, at least about 100 ng/ml, or greater). In another aspect, the level of a circulating and/or systemic sterol and/or oxysterol may be increased to at least about 40 ng per mg of total circulating and/or systemic cholesterol (e.g., at least about 40 ng per mg, at least about 50 ng per mg, at least about 60 ng per mg, at least about 70 ng per mg, at least about 80 ng/mg, at least about 90 ng per mg, or at least about 100 ng per mg of total circulating and/or systemic cholesterol). Generally, the circulating and/or systemic levels of sterols and oxysterols (e.g., after treatment with 2-hydroxypropyl-beta-cyclodextrin) are compared to a baseline level (e.g., a circulating and/or systemic level of the sterol and/or oxysterol in the subject prior to treatment with the 2-hydroxypropyl-beta-cyclodextrin). Non-limiting examples of sterols and oxysterols that may demonstrate increased levels (e.g., in the whole blood, plasma, and/or serum) by administration of 2-hydroxypropyl-beta-cyclodextrin include: 27-hydroxycholesterol, 24-hydroxycholesterol, and 25-hydroxycholesterol. In various aspects, the therapeutically effective amount of 2-hydroxypropyl-beta-cyclodextrin may be an amount sufficient to maintain or sustain the systemic and/or circulating level of the sterol and/or oxysterol for at least 24 hours after treatment (e.g., at least 36 hours, at least 48 hours, at least 72 hours, at least 96 hours).

In a particular aspect, a therapeutically effective amount of 2-hydroxypropyl-beta-cyclodextrin is an amount effective to increase a circulating and/or systemic amount of 27-hydroxycholesterol as compared to a baseline. In some cases, the level of circulating and/or systemic 27-hydroxycholesterol may be increased by at least about 10% as compared to a baseline (e.g., at 24 hours after treatment), for example, increased by at least about 15%, at least about 20%, at least about 25%, at least about 30%, at least about 35%, at least about 40%, at least about 45%, at least about 50%, or greater. In some cases, the level of circulating and/or systemic 27-hydroxycholesterol may be increased to at least about 40 ng/ml, for example, to at least about 50 ng/mL, to at least about 60 ng/ml, to at least about 70 ng/ml, to at least about 80 ng/mL, to at least about 90 ng/ml, to at least about 100 ng/ml, or greater. In a particular embodiment, the level of circulating and/or systemic 27-hydroxycholesterol may be increased to at least about 100 ng/mL. In another aspect, the level of circulating and/or systemic 27-hydroxycholesterol may be increased to at least about 40 ng per mg of total circulating and/or systemic cholesterol, for example, to at least about 50 ng per mg, to at least about 60 ng per mg, to at least about 70 ng per mg, to at least about 80 ng per mg, to at least about 90 ng per mg, or to at least about 100 ng per mg of total circulating and/or systemic cholesterol. In a particular aspect, the level of circulating and/or systemic 27-hydroxycholesterol may be increased to at least about 90 ng per mg of total circulating and/or systemic cholesterol.

Additionally or alternatively, a therapeutically effective amount of 2-hydroxypropyl-beta-cyclodextrin may be an amount effective to increase a circulating and/or systemic amount of 24-hydroxycholesterol as compared to a baseline. In some cases, the level of circulating and/or systemic 24-hydroxycholesterol may be increased by at least about 10% as compared to a baseline (e.g., at 24 hours after treatment), for example, increased at least about 15%, at least about 20%, at least about 25%, at least about 30%, at least about 35%, at least about 40%, at least about 45%, at least about 50%, or greater. In some cases, the level of circulating and/or systemic 24-hydroxycholesterol may be increased to at least about 40 ng/mL, for example, to at least about 50 ng/ml, to at least about 60 ng/ml, to at least about 70 ng/ml, to at least about 80 ng/ml, to at least about 90 ng/mL, to at least about 100 ng/ml, or greater. In a particular embodiment, the level of circulating and/or systemic 24-hydroxycholesterol may be increased to at least about 50 ng/mL. In another aspect, the level of circulating and/or systemic 24-hydroxycholesterol may be increased to at least about 40 ng per mg of total circulating and/or systemic cholesterol, for example, to at least about 50 ng per mg, to at least about 60 ng per mg, to at least about 70 ng per mg, to at least about 80 ng/mg, to at least about 90 ng per mg, or to at least about 100 ng per mg of total circulating and/or systemic cholesterol. In a particular aspect, the level of circulating and/or systemic 24-hydroxycholesterol may be increased to at least about 40 ng per mg of total circulating and/or systemic cholesterol.

A therapeutically effective amount may be an amount of 2-hydroxypropyl-beta-cyclodextrin effective to increase plasma cholesterol crystal dissolution capacity (CCDC) after the administering (e.g., 1 hour after the administering) as compared to prior to the administering. In some cases, the therapeutically effective amount is an amount of 2-hydroxypropyl-beta-cyclodextrin effective to increase plasma CCDC by at least about 10% (e.g., at 1 hour) after the administering as compared to prior to the administering, such as by at least about 15%, at least about 20%, at least about 25%, at least about 30%, at least about 35%, at least about 40%, at least about 45%, at least about 50%, or greater.

A therapeutically effective amount may be an amount of 2-hydroxypropyl-beta-cyclodextrin effective to increase mRNA levels of one or more LXR transcription factor-regulated genes (e.g., ABCA1, ABCG1) after the administering (e.g., 24 hours after the administering) as compared to prior to the administering. In some cases, the therapeutically effective amount is an amount of 2-hydroxypropyl-beta-cyclodextrin effective to increase mRNA levels of ABCA1 and/or ABCG1 by at least about 10% (e.g., at 24 hours) after the administering as compared to prior to the administering, such as by at least about 15%, at least about 20%, at least about 25%, at least about 30%, at least about 35%, at least about 40%, at least about 45%, at least about 50%, or greater.

A therapeutically effective amount can be an amount of 2-hydroxypropyl-beta-cyclodextrin effective to decrease the serum triglyceride level in the subject at least about 5%, at least about 10%, at least about 15%, at least about 20%, at least about 25%, at least about 30%, at least about 35%, at least about 40%, at least about 45%, at least about 50%, as compared to before treatment with 2-hydroxypropyl-beta-cyclodextrin compositions. In some aspects, a therapeutically effective amount can be an amount of 2-hydroxypropyl-beta-cyclodextrin effective to decrease the serum triglyceride level in the subject at least about 5%, at least about 10%, at least about 15%, at least about 20%, at least about 25%, at least about 30%, at least about 35%, at least about 40%, at least about 45%, at least about 50% in 24 hours, in 48 hours, in 72 hours, in a week, in two weeks, in three weeks, in four weeks, in six weeks, in eight weeks after administration. In some aspects, a therapeutically effective amount can be an amount of 2-hydroxypropyl-beta-cyclodextrin effective to decrease the serum triglyceride level in the subject at least about 5%, at least about 10%, at least about 15%, at least about 20%, at least about 25%, at least about 30%, at least about 35%, at least about 40%, at least about 45%, at least about 50%, and maintain the reduced serum triglyceride level for at least 24 hours, at least 48 hours, at least 72 hours, at least a week, at least two weeks, at least three weeks, at least four weeks during and/or after administration.

In some aspects, a therapeutically effective amount of 2-hydroxypropyl-beta-cyclodextrin can be an amount of 2-hydroxypropyl-beta-cyclodextrin effective to maintain a reduced serum triglyceride level following treatment with 2-hydroxypropyl-beta-cyclodextrin. In these maintenance aspects, the therapeutically effective amount can be an amount effective to maintain the serum triglyceride level below at least 250 mg/dL, for example below at least 200 mg/dL, below at least 150 mg/dL, below at least 100 mg/dL or below at least 50 mg/dL. In aspects of maintaining reduced triglyceride levels, the therapeutically effective amount is an amount effective to maintain the serum triglyceride level below at least 250 mg/dL. In other aspects of maintaining reduced triglyceride levels, the therapeutically effective amount is an amount effective to maintain the serum triglyceride level below at least 200 mg/dL. In other aspects of maintaining reduced triglyceride levels, the therapeutically effective amount is an amount effective to maintain the serum triglyceride level below at least 150 mg/dL. In other aspects of maintaining reduced triglyceride levels, the therapeutically effective amount is an amount effective to maintain the serum triglyceride level below at least 100 mg/dL. In other aspects of maintaining reduced triglyceride levels, the therapeutically effective amount is an amount effective to maintain the serum triglyceride level below at least 50 mg/dL.

In maintenance aspects, the reduced serum triglyceride level can be maintained for at least 24 hours, at least 48 hours, at least 72 hours, at least a week, at least two weeks, at least three weeks, at least four weeks during and/or after administration. In aspects of maintaining reduced triglyceride levels, the reduced serum triglyceride level is maintained for at least 24 hours during and/or after administration. In other aspects of maintaining reduced triglyceride levels, the reduced serum triglyceride level is maintained for at least 48 hours during and/or after administration. In other aspects of maintaining reduced triglyceride levels, the reduced serum triglyceride level is maintained for at least 72 hours during and/or after administration. In other aspects of maintaining reduced triglyceride levels, the reduced serum triglyceride level is maintained for at least a week during and/or after administration. In other aspects of maintaining reduced triglyceride levels, the reduced serum triglyceride level is maintained for at least two weeks during and/or after administration. In other aspects of maintaining reduced triglyceride levels, the reduced serum triglyceride level is maintained for at least three weeks during and/or after administration. In other aspects of maintaining reduced triglyceride levels, the reduced serum triglyceride level is maintained for at least four weeks during and/or after administration.

In some aspects, the therapeutically effective amount of 2-hydroxypropyl-beta-cyclodextrin is an amount suitable to achieve the therapeutic effect described herein. In some aspects, the therapeutically effective amount is at least about 50 mg/kg, at least about 100 mg/kg, at least about 200 mg/kg, at least about 300 mg/kg, at least about 400 mg/kg, at least about 500 mg/kg, at least about 600 mg/kg, at least about 700 mg/kg, at least about 800 mg/kg, at least about 900 mg/kg, at least about 1000 mg/kg, at least about 1100 mg/kg, at least about 1200 mg/kg, at least about 1300 mg/kg, at least about 1400 mg/kg, at least about 1500 mg/kg, at least about 1600 mg/kg, at least about 1700 mg/kg, at least about 1800 mg/kg, at least about 1900 mg/kg, or at least about 2000 mg/kg. In some aspects, the therapeutically effective amount of 2-hydroxypropyl-beta-cyclodextrin is at least about 100 mg/kg. In some aspects, the therapeutically effective amount of 2-hydroxypropyl-beta-cyclodextrin is at least about 250 mg/kg. In some aspects, the therapeutically effective amount of 2-hydroxypropyl-beta-cyclodextrin is at least about 500 mg/kg. In some aspects, the therapeutically effective amount of 2-hydroxypropyl-beta-cyclodextrin is at least about 1000 mg/kg. In some aspects, the therapeutically effective amount of 2-hydroxypropyl-beta-cyclodextrin is at least about 1500 mg/kg.

In some aspects, the therapeutically effective amount of 2-hydroxypropyl-beta-cyclodextrin is an amount suitable to achieve the therapeutic effect described herein. In some aspects, the therapeutically effective amount is from about 50 mg/kg to about 2000 mg/kg (e.g., from about 50 mg/kg to about 1000 mg/kg, from about 500 mg/kg to about 1000 mg/kg, from about 500 mg/kg to about 1500 mg/kg, from about 800 mg/kg to about 1500 mg/kg, from about 800 mg/kg to about 1200 mg/kg, from about 1000 mg/kg to about 1500 mg/kg, from about 1000 mg/kg to about 2000 mg/kg. In some aspects, the therapeutically effective amount of 2-hydroxypropyl-beta-cyclodextrin is from about 500 mg/kg to about 1500 mg/kg. In some aspects, the therapeutically effective amount of 2-hydroxypropyl-beta-cyclodextrin is from about 800 mg/kg to about 1200 mg/kg.

In some aspects, the therapeutically effective amount of 2-hydroxypropyl-beta-cyclodextrin is an amount suitable for achieving the therapeutic effect described herein. In some aspects, the therapeutically effective amount is at least about 4 g (e.g., at least about 10 g, at least about 25 g, at least about 50 g, at least about 75 g, at least about 100 g, at least about 125 g, at least about 150 g, at least about 175 g, at least about 200 g, at least about 250 g). In some aspects, the therapeutically effective amount of 2-hydroxypropyl-beta-cyclodextrin may be from about 4 g to about 250 g (e.g., from about 4 g to about 200 g, from about 4 g to about 150 g, from about 4 g to about 100 g, from about 4 g to about 50 g, from about 50 g to about 250 g, from about 50 g to about 200 g, from about 50 g to about 150 g, from about 50 g to about 100 g, from about 100 g to about 250 g, from about 100 g to about 200 g). The total amount of 2-hydroxpropyl-beta-cyclodextrin administered (e.g., in a single dose administration, e.g., in a therapeutically effective amount) may depend on a number of factors, including, without limitation, the subject's age, gender, weight, and the like.

In some aspects, the therapeutically effective amount of 2-hydroxypropyl-beta-cyclodextrin is an amount sufficient to achieve a whole blood, serum, and/or plasma concentration of 2-hydroxypropyl-beta-cyclodextrin suitable for achieving the therapeutic effect described herein. In some aspects, the whole blood, serum, and/or plasma concentration is at least about 0.1 mM (e.g., at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1.0 mM, at least about 1.5 mM, at least about 2.0 mM, at least about 2.5 mM, or at least about 3 mM). The therapeutically effective amount of 2-hydroxypropyl-beta-cyclodextrin may be an amount sufficient to achieve a whole blood, serum, and/or plasma concentration of 2-hydroxypropyl-beta-cyclodextrin of about 0.6 mM to about 3 mM (e.g., about 0.6 mM to about 2 mM, about 0.6 mM to about 1 mM, about 1 mM to about 3 mM, about 1 mM to about 2 mM, about 2 mM to about 3 mM).

The methods disclosed herein may further comprise administering, at a first time point, a therapeutically effective first amount of 2-hydroxypropyl-beta-cyclodextrin to a subject, and administering, at a second time point, a therapeutically effective second amount of 2-hydroxypropyl-beta-cyclodextrin to the subject. The second time point can be at least 1 day, 2 days, 3 days, 4 days, 5 days, or 6 days after the first time point. The second time point can be at least 1 week after the first time point (e.g., 2 weeks, 3 weeks, 4 weeks, 5 weeks, 6 weeks, 7 weeks, 8 weeks, 9 weeks, or 10 weeks after the first time point). In some cases, the second time point is one or more week after the first time point, two weeks after the first time point, or one or more month after the first time point. In some aspects, the administering may be by intravenous administration.

In some cases, the second time point may be determined based on one or more indicators that an additional dose of drug would be beneficial to the subject. For example, the second time point may be administered after the therapeutic benefit of the first dose has diminished or has started to diminish. The second time point can be determined based on, e.g., levels of circulating and/or systemic 27-hydroxycholesterol, levels of circulating and/or systemic 24S-hydroxycholesterol levels, transcription levels of ATP-binding cassette subfamily A member 1 (ABCA1), transcription levels of ATP-binding cassette subfamily G member 1 (ABCG1), epigenetic (chromatin) signature of peripheral blood mononuclear cells (PBMCs), triglyceride levels, total cholesterol levels, very low-density lipoprotein (vLDL) level, low density lipoprotein (LDL) level, high density lipoprotein (HDL) level, serum or plasma cholesterol crystal dissolution, levels of pro-inflammatory mediators (e.g., interleukin-1b (IL-1b), interleukin-6 (IL-6), interleukin-18 (IL-18)).

In various aspects, the subject can be a human. In some cases, the subject may be of any age that is at risk of or more prone to developing hypertriglyceridemia. The subject may be at least 30 years old (e.g., at least 40, at least 50 at least 60, at least 70, at least 80, at least 90 years old). The subject may be less than 30 years old (e.g., less than 20 years old, less than 15 years old, less than 10 years old, or less than 5 years old). The subject can be diagnosed with hypertriglyceridemia. The subject can be diagnosed with hypertriglyceridemia, for example, if the subject has greater than 100 mg/dL, greater than 150 mg/dL or greater than 200 mg/dL of triglycerides in the blood. The subject can be diagnosed with hypertriglyceridemia, for example, if the subject has greater than 100 mg/dL of triglycerides in the blood. The subject can be diagnosed with hypertriglyceridemia, for example, if the subject has greater than 150 mg/dL (1.7 millimoles/liter) of triglycerides in the blood. The subject can be diagnosed with hypertriglyceridemia, for example, if the subject has greater than 200 mg/dL of triglycerides in the blood. The subject can be diagnosed with acute coronary syndrome (ACS) or chronic coronary syndrome (CCS) (e.g., as defined by the European Society of Cardiology).

Hypertriglyceridemia can be diagnosed via blood test (e.g., fasting triglyceride level >200 mg/dL without an accompanying elevation in LDL-C). In some cases, symptoms associated with hypertriglyceridemia or its associated diseases can be diagnosed via angiogram, cholesterol test, a computed tomography (CT) scan, Duplex scanning, an echocardiogram, an electrocardiogram (ECG or EKG), exercise stress test, an intravascular ultrasound, a magnetic resonance imaging (MRI) scan, a positron emission tomography (PET) scan, an optical coherence tomography (OCT) scan, a pharmacologic stress test, symptoms/medical history (e.g., patient-reported symptoms), fat attenuation index (FAI), blood tests, or a combination thereof. The subject can have a symptom associated with hypertriglyceridemia. The symptom associated with hypertriglyceridemiacan be chest pain (e.g., angina), shortness of breath, fatigue, confusion, muscle weakness, skin symptoms (e.g., eruptive xanthoma), eye abnormalities (e.g., lipemia retinalis), hepatosplenomegaly (e.g., enlargement of the liver and spleen, neurological symptoms, abdominal pain, or a combination thereof. The subject can be at risk of developing hypertriglyceridemia. A subject at risk of developing hypertriglyceridemia can have at least one hypertriglyceridemia risk factor. In some aspects, hypertriglyceridemia risk factors include, without limitation, being overweight or obese, high blood pressure, high cholesterol level, diabetes, lack of physical activity, one or more co-morbidities (e.g., smoking, renal disease, rheumatoid disease), excessive alcohol consumption, kidney failure, nephrotic syndrome, genetic predisposition, lipoprotein lipase deficiency, lysosomal acid lipase deficiency, hypothyroidism, lupus, glycogen storage disease, propofol, HIV medication, and the use of chemotherapeutic agents. The subject can have at least a 10%, 20%, 30%, 40%, 50%, 60%, 70%, 80%, or 90% stenosis of an artery and/or a vein. The subject can have at least a 50% stenosis of an artery and/or a vein. The artery can be, e.g., a coronary artery, a cerebral artery, a peripheral artery, the aorta. The subject may have one or more atherosclerotic plaques having a high cholesterol and/or high lipid content (e.g., as measured by optical coherence tomography (OCT)). The subject may have one or more plaques having a low calcium score (e.g., as measured by a computed tomography (CT) scan). The subject may have stable or unstable CAD or hypertriglyceridemia. The subject may have acute coronary syndrome (ACS) or chronic coronary syndrome (CCS) (e.g., as defined by the European Society of Cardiology). The subject may be treated (e.g., by the methods described herein) after (e.g., immediately after) having a myocardial infarction. The subject may have a thickening of the arterial wall (tunica media). The subject may be treated (e.g., by the methods described herein) after undergoing chemotherapy (e.g., the subject may have an increased risk of or may have developed hypertriglyceridemia due to the use of chemotherapeutic agents).

The methods disclosed herein can be used to treat and/or prevent hypertriglyceridemia. The methods disclosed herein can be used to treat and/or prevent atherosclerosis or cardiovascular disease that may be caused by hypertriglyceridemia.

In some aspects, the methods described herein causes a reduction in the size of atherosclerotic plaques (e.g., cholesterol-rich plaques and/or lipid-rich plaques) in the subject suffering from or suspected to suffer from hypertriglyceridemia. The plaques may have a high cholesterol and/or high lipid content. The cholesterol and/or lipid content may be measured by, e.g., optical coherence tomography (OCT). The plaques may have a low calcium content. The plaques may have a low calcium score on a computed tomography (CT) scan. In some cases, the size of an atherosclerotic plaque may be reduced relative to the size of the atherosclerotic plaque prior to the treating. In some aspects, the size of an atherosclerotic plaque may be reduced by at least about 0.5%. In some aspects, the size of an atherosclerotic plaque may be reduced by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or greater.

In some aspects, the methods described herein cause a decrease in and/or prevent the progression and/or development of hypertriglyceridemia in the subject. In some aspects, the methods described herein prevent the progression of hypertriglyceridemia. For example, the methods described herein prevent an increase in the size of atherosclerotic plaques, prevent an increase in plaque volume, prevent an increase in the amount of the coronary arterial surface covered by plaques, prevent thickening of the atherosclerotic plaques, prevent an increase in stenosis of an artery or vein, prevent or reduce thickening of an arterial wall, and/or prevent or reduce vascular calcification. In some cases, the methods described herein mediate the regression of already-developed atherosclerotic plaques in the subject. For example, the methods described herein reduce the size and/or number of atherosclerotic plaques already-developed in the subject, and/or reduce the size of the necrotic core of an atherosclerotic plaque.

The treating may result in an increase in flow-mediated vasodilation (FMD) of the brachial artery. The increase in flow-mediated vasodilation (FMD) can occur at least 7 days after the treating. Flow-mediated vasodilation (FMD) can be measured by, e.g., ischemia-induced endothelial-dependent vasodilation. In some aspects, flow-mediated vasodilation (FMD) is determined using high resolution ultrasonography. In some cases, the treating may result in an increase in sterol and/or oxysterol levels (e.g., 27-hydroxycholesterol, 24-hydroxycholesterol) in whole blood, serum, plasma, or any combination thereof. In some cases, the treating may result in the dissolution of cholesterol crystals e.g., present in atherosclerotic plaques. Cholesterol crystal dissolution can be measured by, e.g., a cholesterol crystal dissolution capacity assay. In some cases, the treating may result in an increase in gene expression of a liver X receptor (LXR)-regulated gene. The increase in the LXR-regulated gene can be in, e.g., peripheral blood mononuclear cells (PBMCs) of the subject. The LXR-regulated gene can be, e.g., ATP-binding cassette subfamily A member 1 (ABCA1), ATP-binding cassette subfamily G member 1 (ABCG1), fatty acid synthase (FAS), apolipoprotein E (APOE), or a combination thereof. In some cases, the treating may result in an increase in the phagocytic activity of PBMCs. In some cases, the treating may result in an increase or a decrease in a level of a lipid in a biological sample from the subject. The lipid can be a triglyceride, LDL-cholesterol, HDL-cholesterol, or apolipoprotein A1 (ApoA1). The treating can result in a decrease in a level of triglycerides in the biological sample. The treating can result in a decrease of LDL-cholesterol in the biological sample. The treating can result in an increase in a level of HDL-cholesterol in the biological sample. The treating can result in an increase in a level of ApoA1 in the biological sample. The biological sample can be blood (e.g., whole blood, serum, plasma). In some cases, the treating may result in a decrease in serum markers of inflammation and myocardial damage and/or an increase in serum markers of anti-inflammation. The serum marker can be interleukin (IL)-1 beta (IL-1beta), interleukin-1 receptor antagonist (IL-Ira), interleukin-1 alpha (IL-la), interleukin-6 (IL-6), highly sensitive C-reactive protein (hsCRP), Troponin, creatine kinase (CK), creatine kinase-MB (CK-MB), N-terminal pro-B-type natriuretic peptide (NT-pro-BNP). In some cases, the treating may result in a decrease in complement activation. In some cases, the treating may result in a decrease in a risk of mortality and/or all-cause mortality (ACM) of the subject. In some aspects, the risk of mortality of the subject is decreased by 25%, 50%, 75%, or 90%. In some aspects, the risk of mortality of the subject is decreased for at least 1 year, 2 years, or 3 years after the administering. In some cases, the treating may result in a decrease in a risk of myocardial infarction in the subject. In some aspects, the risk of myocardial infarction in the subject is decreased by 25%, 50%, 75%, or 90%. In some aspects, the risk of myocardial infarction in the subject is decreased for at least 1 year, 2 years, or 3 years after the administering. In some cases, the treating may result in a decrease in a risk of major or minor stroke in the subject. In some aspects, the risk of major or minor stroke in the subject is decreased by 25%, 50%, 75%, or 90%. In some aspects, the risk of major or minor stroke in the subject is decreased for at least 1 year, 2 years, or 3 years after the administering. In some cases, the treating may result in a decrease in a blood pressure of the subject. In some aspects, the blood pressure of the subject is decreased by at least 5%, 10%, 15%, 20%, 25%, or 30%. The decrease in blood pressure can comprise a decrease in systolic blood pressure, diastolic blood pressure, or a combination thereof. In some cases, the treating may result in a decrease in a risk of a major adverse cardiovascular event (MACE) in the subject. In some aspects, a major adverse cardiovascular event comprises heart failure, re-infarction, recurrent angina pain, re-hospitalization for cardiovascular-related illness, repeat percutaneous coronary intervention (PCI), coronary artery bypass grafting, coronary revascularization, stroke, all-cause mortality (ACM), or a combination thereof. In some aspects, the risk of MACE in the subject is decreased by 25%, 50%, 75%, or 90%. In some aspects, the risk of MACE in the subject is decreased for at least 1 year, 2 years, or 3 years after the administering. In some cases, the treating may result in improved erectile dysfunction, in correlation to CAD or hypertriglyceridemia severity.

In some cases, the treating may result in a level of a liver enzyme less than 2.5 times up to a normal level of the liver enzyme. The liver enzyme can be alanine aminotransferase (ALT), aspartate aminotransferase (AST), or the combination thereof. The liver enzyme can be alanine aminotransferase (ALT), aspartate aminotransferase (AST), alkaline phosphatase (ALP), 5′ nucleotidase, gamma-glutamyl transpeptidase (GGT), or a combination thereof. In some aspects, the normal level of the liver enzyme is a level of the liver enzyme in the absence of damage to the liver. In some cases, the treating may result in serum creatinine levels less than 0.3 mg/dl. The treating may result in serum creatinine levels less than 1.3, 1.0, 0.75, 0.5, or 0.3 mg/dl. In some cases, the treating may result in no substantial loss of sensorineural hearing.

### Pharmaceutical Compositions

Disclosed herein, in certain aspects, are pharmaceutical compositions comprising an amount of 2-hydroxypropyl-beta-cyclodextrin effective to treat hypertriglyceridemia in a human; and an excipient. The excipient can be a pharmaceutically acceptable excipient.

The pharmaceutical composition may comprise an amount of 2-hydroxypropyl-beta-cyclodextrin effective to increase a circulating and/or systemic level of one or more oxysterol in a subject by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase plasma cholesterol crystal dissolution capacity (CCDC) by at least about 10% (e.g., at 1 hour) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase mRNA levels of one or more LXR transcription factor-regulated genes (e.g., ABCA1 and/or ABCG1) by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to decrease serum triglyceride levels by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to maintain the serum triglyceride levels that is decreased at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater, for at least 12 hours, at least 24 hours, at least 48 hours, at least 72 hours, at least 7 days, or at least 2 weeks.

The excipient may comprise a tonicity adjusting agent, a preservative, a solubilizing agent, a buffer, a solution (e.g., an IV solution), or any combination thereof. The tonicity adjusting agent can be dextrose, glycerol, sodium chloride, glycerin, mannitol, or a combination thereof. The preservative can be an antioxidant, an antimicrobial, a chelating agent, or a combination thereof. The antioxidant can be ascorbic acid, acetylcysteine, a sulfurous acid salt (e.g., bisulfite, metabisulfite), a monothioglycerol, or a combination thereof. The antimicrobial can be a phenol, meta-cresol, benzyl alcohol, paraben, benzalkonium chloride, chlorobutanol, thimerosal, phenylmercuric salts (e.g., acetate, borate, nitrate), or a combination thereof. The chelating agent can be calcium disodium ethylenediaminetetraacetic acid (EDTA), disodium EDTA, sodium EDTA, calcium versetamide sodium, calteridol, diethylenetriaminepenta acetic acid (DTPA), or a combination thereof. The solubilizing agent can be a surfactant or a co-solvent. The surfactant can be polyoxyethylene sorbitan monooleate (Tween 80), sorbitan monooleate polyoxyethylene sorbitan monolaurate (Tween 20), lecithin, polyoxyethylene-polyoxypropylene copolymers (Pluronics), or a combination thereof. The co-solvent can be propylene glycol, glycerin, ethanol, polyethylene glycol (PEG), sorbitol, dimethylacetamide, Cremophor EL, or a combination there. The polyethylene glycol can be PEG 300, PEG 400, PEG 600, PEG 3350, or PEG 4000. The buffer can comprise sodium acetate, acetic acid, glacial acetic acid, ammonium acetate, ammonium sulfate, ammonium hydroxide, arginine, aspartic acid, benzene sulfonic acid, benzoate sodium, benzoic acid, sodium bicarbonate, boric acid, sodium boric acid, sodium carbonate, citrate acid, sodium citrate, disodium citrate, trisodium citrate, diethanolamine, glucono delta lactone, glycine, glycine HCl, histidine, histidine HCl, hydrochloric acid, hydrobromic acid, lysine, maleic acid, meglumine, methanesulfonic acid, monoethanolamine, phosphate acid, monobasic potassium, dibasic potassium, monosodium phosphate, disodium phosphate, trisodium phosphate, sodium hydroxide, succinate sodium, sulfuric acid, tartarate sodium, tartaric acid, tromethamine (Tris), or a combination thereof.

The pharmaceutical composition can comprise at least about 4, at least about 10, at least about 50, at least about 100, at least about 150, at least about 200, or at least about 250 g of 2-hydroxypropyl-beta-cyclodextrin. In some aspects, the pharmaceutical composition comprises at least about 4 g of 2-hydroxypropyl-beta-cyclodextrin. In some aspects, the pharmaceutical composition comprises at least about 50 g of 2-hydroxypropyl-beta-cyclodextrin. In some aspects, the pharmaceutical composition comprises at least about 100 g of 2-hydroxypropyl-beta-cyclodextrin. In some aspects, the pharmaceutical composition comprises at least about 200 g of 2-hydroxypropyl-beta-cyclodextrin. In some aspects, the pharmaceutical composition comprises from about 4 g to about 250 g of 2-hydroxypropyl-beta-cyclodextrin (e.g., from about 4 g to about 100 g, from about 4 g to about 50 g, from about 50 g to about 150 g, from about 50 g to about 250 g, from about 100 g to about 200 g, from about 100 g to about 250 g, from about 150 g to about 250 g.)

The pharmaceutical composition can be formulated for single dose administration. The pharmaceutical composition can be formulated for intravenous administration. The pharmaceutical composition can be formulated to be isotonic.

### Kits

Further provided herein are kits. In some cases, the kits include one or more container (e.g., a vial, a flask, a jar, a tube, an ampoule, etc.) containing one or more pharmaceutical compositions provided herein (e.g., 2-hydroxypropyl-beta-cyclodextrin and a pharmaceutically acceptable excipient). In some cases, the kit comprises more than one container (e.g., two, three, four, five, six, seven, eight, nine, ten, or more containers). In some cases, at least one of the one or more container is an IV infusion bag. The one or more container may include a single dosage of the pharmaceutical composition, or multiple dosages (e.g., two, three, four, five, six, seven, eight, nine, ten, or more) of the pharmaceutical composition. In some cases, the one or more container contains a concentrated amount of the pharmaceutical composition, which is subsequently diluted, prior to administration, to achieve an effective dosage. The dosage may be any amount as described herein, effective to treat one or more indications described herein. The kit may further comprise one or more additional components for IV infusion of the pharmaceutical composition. In some cases, the kit comprises an IV infusion bag. In some cases, the kit comprises one or more solutions (e.g., saline) for mixing and/or diluting the pharmaceutical composition. In some cases, the kit comprises one or more of a catheter, a tubing, a syringe, and a needle. The kit may further comprise instructions, e.g., for administering the pharmaceutical composition to a subject for the use of treating any indication described herein (e.g., for the treatment of hypertriglyceridemia in a subject (e.g., human individual) and/or reducing or inhibiting the development of cholesterol rich plaque in a subject (e.g., human individual) suffering from or suspected to suffer from hypertriglyceridemia). The kit may be provided in a box, a bag, or any other suitable container.

In some aspects, the kit may comprise one or more additional active pharmaceutical ingredient (e.g., therapeutic compounds, drugs, etc.). In some cases, the kit may comprise a single container containing a pharmaceutical composition of the disclosure (e.g., 2-hydroxypropyl-beta-cyclodextrin and a pharmaceutically acceptable excipient) and the one or more additional active pharmaceutical ingredient. In other cases, the kit may comprise a first container containing a pharmaceutical composition of the disclosure (e.g., 2-hydroxypropyl-beta-cyclodextrin and a pharmaceutically acceptable excipient) and a second container containing the one or more additional active pharmaceutical ingredient.

### Example

### Example 1.2-Hydroxypropyl-Beta-Cyclodextrin (HPBCD) Effectively Decreases Serum Triglyceride Levels in LDLR^{-/-}Mouse Model

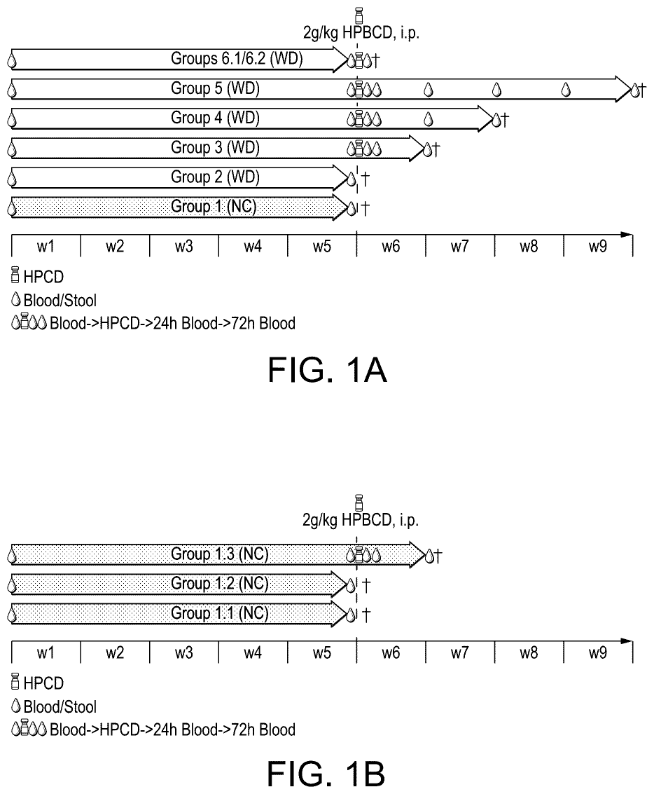

LDL receptor deficient (LDLR^{-/-}) mice were divided into 6 Groups. Prior to the administration of 2 g/kg of HPBCD, Group 1 was fed with normal chow diet (NC) for 5 weeks and Group 2-Group 6 were fed with high-fat (42%), high-cholesterol (1.2%) Western diet (WD) for various durations up to 9 weeks. Experimental scheme and diet schedule is described in FIG. 1A.

Mice of Group 1 were further categorized into 3 sub-Groups (e.g., Group 1.1, Group 1.2, and Group 1.3) according to the administration of HPBCD as shown in FIG. 1B.

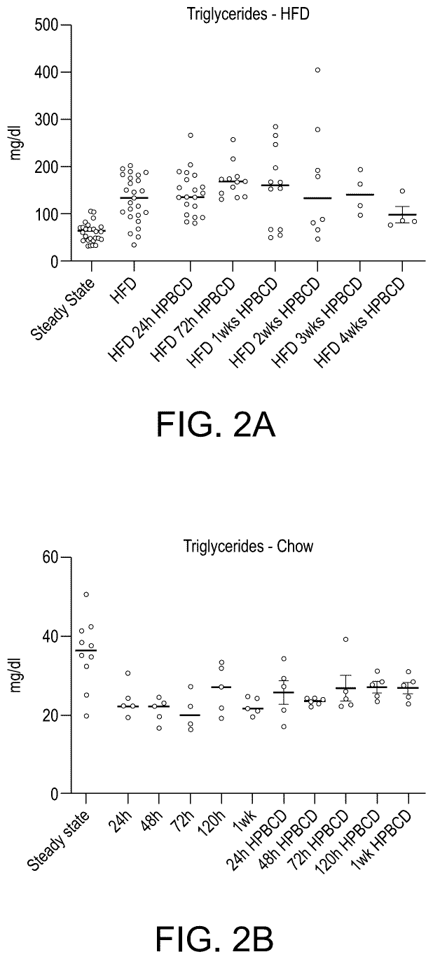

Blood samples were taken from the mouse models, after 5 weeks of feeding and at a certain period of time (e.g., 1 week, 2 weeks, 3 weeks, 4 weeks after 2 g/kg HPBCD treatment), for measurement of triglyceride levels in each model Group. Compared with NC-fed controls (Group 1) at steady state of triglyceride level, WD-fed mice (Group 2 to Group 6) showed increasing levels of triglyceride. However, triglyceride levels of WD-fed mice decreased over time after the administration of HPBCD, indicating that HPBCD can be administered to treat hypertriglyceridemia, and/or reducing or preventing symptoms of hypertriglyceridemia. (FIG. 2A). The similar trend of triglyceride level was observed in NC-fed mice (FIG. 2B). The triglyceride level of NC-fed mice also decreased from 24 hours after the administration of HPBCD.

### Example 2.2-hydroxypropyl-beta-cyclodextrin (HPBCD) safely and effectively decreases serum triglyceride levels in humans

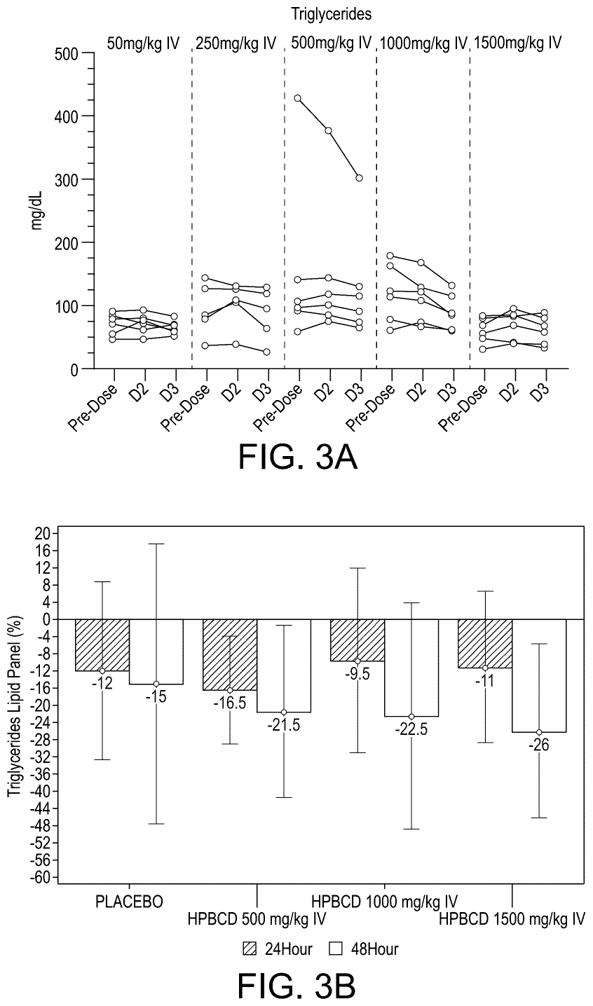

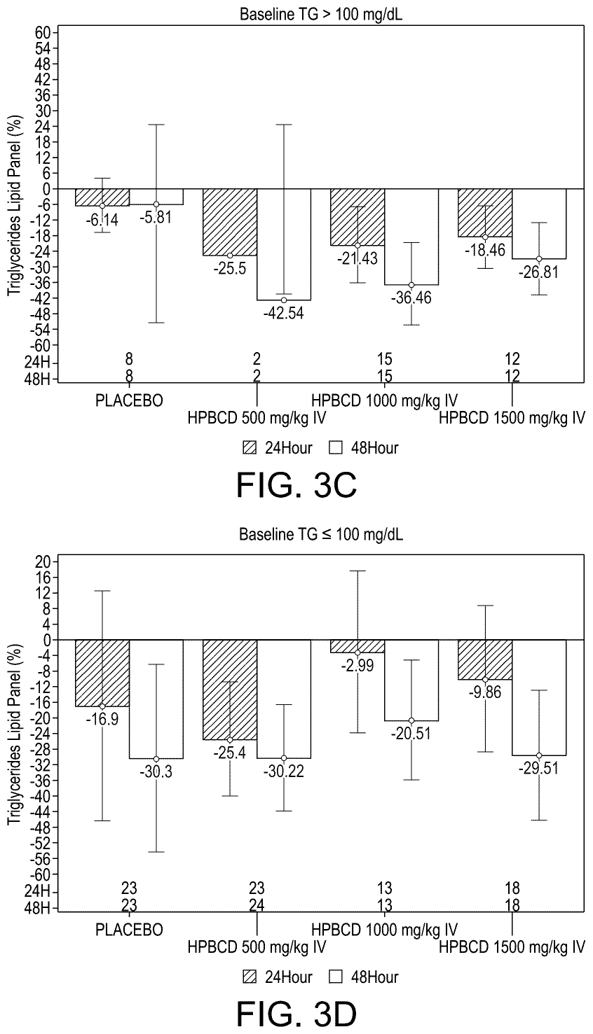

Twenty-nine volunteers were divided into five groups. Each group was administered an intravenous dose of 50 mg/kg, 250 mg/kg, 500 mg/kg, 1000 mg/kg or 1500 mg/kg of HPBCD as shown in FIG. 3A. Blood samples were taken immediately prior to injection to establish a baseline triglyceride concentration, and at 24 hours (D2) and 48 hours (D3) post injection for measurement of triglyceride levels. Triglyceride levels did not significantly increase following administration of any tested dose confirming the safety of HPBCD. Overall, triglyceride levels of participants decreased, particularly evident in participants with higher baseline triglyceride concentrations.

In a separate study, volunteers were divided into four groups. Each group was administered an intravenous dose of placebo or an intravenous dose of 500 mg/kg, 1000 mg/kg or 1500 mg/kg of HPBCD as shown in FIG. 3B. Blood samples were taken immediately prior to injection to establish a baseline triglyceride concentration, and at 24 hours (left bar) and 48 hours (right bar) post injection. Triglyceride levels are presented as the median average percentage change from baseline. In line with the data presented in FIG. 3A, triglyceride levels did not increase following administration of any tested dose.

The data in FIG. 3B were divided into two groups for further analysis. Group 1 consisted of the portion of the cohort which had baseline triglyceride levels greater than 100 mg/dL. Group 2 had baseline triglyceride levels less than or equal to 100 mg/dL. The average percentage change of triglyceride levels from baseline of Group 1 and Group 2 is shown in FIG. 3C and FIG. 3D respectively. A greater reduction in triglyceride levels was observed in participants in Group 1 than those in Group 2. This is consistent with the marked reduction (˜30%) in triglyceride levels observed for the one participant in FIG. 3A with a baseline triglyceride level of approximately 425 mg/dL. Overall, the data demonstrate that HPBCD effectively decreases serum triglyceride levels in humans, particularly in those with higher baseline triglyceride levels.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating hypertriglyceridemia in a subject, the method comprising: administering a therapeutically effective amount of 2-hydroxypropyl-beta-cyclodextrin to the subject to treat hypertriglyceridemia and/or to reduce or prevent symptoms of hypertriglyceridemia.

2. The method of claim 1, wherein the hypertriglyceridemia is caused by high triglyceride levels in the subject, overeating, obesity, diabetes and/or insulin resistance, excessive alcohol consumption, kidney failure, nephrotic syndrome, genetic predisposition, lipoprotein lipase deficiency, lysosomal acid lipase deficiency, hypothyroidism, lupus, glycogen storage disease, propofol, and/or HIV medication.

3. A method of reducing symptoms of or inhibiting the development of hypertriglyceridemia in a subject, the method comprising administering a therapeutically effective amount of 2-hydroxypropyl-beta-cyclodextrin to the subject, the therapeutically effective amount being:
(a) an amount effective to increase a circulating and/or systemic level of one or more oxysterol in the subject by at least about 10% after the administering as compared to prior to the administering;
(b) an amount effective to increase plasma cholesterol crystal dissolution capacity (CCDC) by at least about 10% after the administering as compared to prior to the administering;
(c) an amount effective to increase a level of ABCA1 and/or ABCG1 by at least about 10% after the administering as compared to prior to the administering;
(d) about 50 mg/kg to about 2,000 mg/kg
(e) an amount effective to decrease the amount of serum triglyceride by at least 10% after the administering as compared to prior to the administering; or
(f) any combination thereof,
thereby reducing symptoms of or inhibiting the development of hypertriglyceridemia in the subject.

4. The method of any one of the preceding claims, wherein the method comprises maintaining a reduced serum triglyceride level below at least 250 mg/dL for at least 24 hours, at least 48 hours, at least 72 hours, at least a week, at least two weeks, at least three weeks or at least four weeks after administration.

5. The method of claim 4, wherein the method comprises maintaining a reduced serum triglyceride level below at least 150 mg/dL for at least two weeks.

6. The method of any one of the preceding claims, wherein the therapeutically effective amount is about 2 g to about 250 g of the 2-hydroxypropyl-beta-cyclodextrin.

7. The method of any one of the preceding claims, wherein the therapeutically effective amount is an amount sufficient to achieve a serum, plasma, and/or whole blood concentration of 2-hydroxpropyl-beta-cyclodextrin of about 0.6 mM to about 3 mM.

8. The method of any one of the preceding claims, wherein the therapeutically effective amount is an amount effective to increase a circulating and/or systemic level of one or more oxysterol in the subject by at least about 10% after the administering as compared to prior to the administering.

9. The method of claim 8, wherein the circulating and/or systemic levels comprise serum, plasma, and/or whole blood levels.

10. The method of claim 8 or 9, wherein the one or more oxysterols are selected from the group consisting of: 27-hydroxycholesterol and 24-hydroxycholesterol.

11. The method of any one of claims 8-10, wherein the at least about 10% comprises at least about 15%, at least about 20%, at least about 30%, at least about 40%, or at least about 50%.

12. The method of any one of claims 8-11, wherein the therapeutically effective amount is an amount effective to increase a circulating and/or systemic level of one or more oxysterol to about 40 ng/ml or greater.

13. The method of any one of claims 8-12, wherein the therapeutically effective amount is an amount effective to increase a circulating and/or systemic level of one or more oxysterol to at least about 40 ng per mg of total circulating and/or systemic cholesterol.

14. The method of any one of claims 8-13, wherein the one or more oxysterol comprises 27-hydroxycholesterol.

15. The method of claim 14, wherein the therapeutically effective amount is an amount effective to increase the circulating and/or systemic level of 27-hydroxycholesterol to at least about 100 ng/mL.

16. The method of claim 14 or 15, wherein the therapeutically effective amount is an amount effective to increase the circulating and/or systemic level of 27-hydroxycholesterol to at least about 90 ng per mg of total circulating and/or systemic cholesterol.

17. The method of any one of claims 8-16, wherein the therapeutically effective amount is an amount sufficient to sustain the circulating and/or systemic level of the one or more oxysterol for at least 24 hours.

18. The method of any one of the preceding claims, wherein the therapeutically effective amount is an amount effective to increase plasma cholesterol crystal dissolution capacity (CCDC) by at least about 10% after the administering as compared to prior to the administering.

19. The method of any one of the preceding claims, wherein the therapeutically effective amount is an amount effective to increase a level of ABCA1 and/or ABCG1 by at least about 10% at after the administering as compared to prior to the administering.

20. The method of any one of the preceding claims, wherein the therapeutically effective amount is an amount effective to decrease the amount of serum triglyceride by at least 10% after the administering as compared to prior to the administering.

21. The method of any one of the preceding claims, wherein the therapeutically effective amount is about 50 mg/kg to about 2,000 mg/kg.

22. The method of claim 21, wherein the therapeutically effective amount is at least about 100 mg/kg.

23. The method of claim 21 or 22, wherein the therapeutically effective amount is at least about 250 mg/kg.

24. The method of any one of claims 21-23, wherein the therapeutically effective amount is at least about 500 mg/kg.

25. The method of any one of claims 21-24, wherein the therapeutically effective amount is at least about 1,000 mg/kg.

26. The method of any one of claims 21-25, wherein the therapeutically effective amount is at least about 1,500 mg/kg.

27. The method of any one of claims 20-26, wherein the therapeutically effective amount is about 500 mg/kg to about 1,500 mg/kg.

28. The method of any one of claims 20-27, wherein the therapeutically effective amount is about 800 mg/kg to about 1,200 mg/kg.

29. The method of any one of the preceding claims, wherein the subject is an human individual.

30. The method of any one of the preceding claims, wherein the administering further comprises: (i) administering, at a first time point, a therapeutically effective first dose of 2-hydroxypropyl-beta-cyclodextrin to the subject; and (ii) administering, at a second time point, a therapeutically effective second dose of 2-hydroxypropyl-beta-cyclodextrin to the subject.

31. The method of claim 30, wherein the second time point is at least 1 week after the first time point.

32. The method of claim 30 or 31, wherein the second time point is at least 2 weeks after the first time point.

33. The method of any one of claims 30-32, wherein the second time point is at least one month after the first time point.

34. The method of any one of the preceding claims, wherein the treating comprises decreasing or preventing progression and/or development of hypertriglyceridemia in the subject.

35. The method of any one of the preceding claims, wherein the treating comprises mediating the regression of elevated serum triglyceride level in the subject.

36. The method of any one of the preceding claims, wherein the treating results in one or more of the following:
a) liver enzyme (e.g., ALT, AST) levels less than 2.5 times to normal;
b) serum creatinine levels less than 0.3 mg/dl; or
c) no substantial loss of sensorineural hearing.

37. The method of any one of the preceding claims, wherein the administering is by intravenous administration.

38. A pharmaceutical composition comprising: an amount of 2-hydroxypropyl-beta-cyclodextrin effective to treat hypertriglyceridemia in a subject, and a pharmaceutically acceptable excipient.

39. A pharmaceutical composition comprising: about 4 g to about 250 g of 2-hydroxypropyl-beta-cyclodextrin and a pharmaceutically acceptable excipient.

40. The pharmaceutical composition of claim 38 or 39, wherein the amount of 2-hydroxypropyl-beta-cyclodextrin is an amount effective to increase a circulating and/or systemic level of one or more oxysterol in the subject by at least about 10% after administering the pharmaceutical composition to the subject.

41. The pharmaceutical composition of any one of claims 38-40, wherein the amount of 2-hydroxypropyl-beta-cyclodextrin is an amount effective to increase plasma cholesterol crystal dissolution capacity (CCDC) by at least about 10% after administering the pharmaceutical composition to the subject.

42. The pharmaceutical composition of any one of claims 38-41, wherein the amount of 2-hydroxypropyl-beta-cyclodextrin is an amount effective to increase a level of ABCA1 and/or ABCG1 by at least about 10% after administering the pharmaceutical composition to the subject.

43. The pharmaceutical composition of any one of claims 38-42, wherein the amount of 2-hydroxypropyl-beta-cyclodextrin is an amount effective to decrease the amount of serum triglyceride by at least 10% after administering the pharmaceutical composition to the subject.

44. The pharmaceutical composition of any one of claims 38-43, wherein the amount of 2-hydroxypropyl-beta-cyclodextrin is an amount effective to maintain reduced serum triglyceride levels below at least 250 mg/dL for at least 24 hours, at least 48 hours, at least 72 hours, at least a week, at least two weeks, at least three weeks or at least four weeks after administering the pharmaceutical composition to the subject.

45. The pharmaceutical composition of claim 44, wherein the amount of 2-hydroxypropyl-beta-cyclodextrin is an amount effective to maintain a reduced serum triglyceride level below at least 150 mg/dL for at least two weeks after administering the pharmaceutical composition to the subject.

46. The pharmaceutical composition of any one of claims 38-45, formulated for single dose administration.

47. The pharmaceutical composition of any one of claims 38-46, formulated for intravenous administration.

48. A kit comprising:
(a) one or more container; and
(b) the pharmaceutical composition of any one of claims 38-47, wherein the pharmaceutical composition is contained within the one or more container.

49. The kit of claim 48, further comprising (c) instructions for use of the pharmaceutical composition for the treatment of hypertriglyceridemia in a subject and/or reducing or inhibiting the development of hypertriglyceridemia or symptoms thereof in a subject.

50. The kit of claim 48 or 49, wherein at least one of the one or more container is an IV infusion bag.

51. The kit of any one of claims 48-50, wherein the one or more container comprises a single container comprising the pharmaceutical composition and one or more additional active pharmaceutical ingredients.

52. The kit of any one of claims 48-50, wherein the one or more container comprises a first container containing the pharmaceutical composition and a second container containing one or more additional active pharmaceutical ingredients.

53. The kit of any one of claims 48-52, further comprising one or more additional components selected from the group consisting of: an IV infusion bag, a catheter, tubing, a needle, a syringe, a solution, and any combination thereof.
